(19) United States
(12) Patent Application Publication
(10) Pub. No.: US 2006/0246104 A1
(43) Pub. Date: Nov. 2, 2006

(54) Stable rgd peptidomimetic composition

(76) Inventors: Stephen P. Massia, Phoenix, AZ (US); Gholamreza Ehteshami, Scottsdale, AZ (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/549,518

(22) PCT Filed: Apr. 16, 2004
(86) PCT No.: PCT/US04/11929

§ 371 Date: Sep. 14, 2005

Related U.S. Application Data
(60) Provisional application No. 60/463,620, filed on Apr. 16, 2003.

(51) Int. Cl. A61K 38/04; C07K 5/04; A61F 2/00

(52) U.S. Cl. 424/423; 514/19; 562/560

(57) ABSTRACT

Disclosed herein are a chemical composition of 2-amino-6-[(2-amino-5{guanidine}pentanoyl)amino]hexanoic acid (AAGPAHA) and a method of making it. The pharmaceutical composition includes AAGPAHA and a pharmaceutical excipient. Such a composition is used in cell attachment in culture, as a coating of biomaterials and implants. The pharmaceutical composition of AAGPAHA can be used to treat cancer, thrombosis, osteoporosis, retinopathy, renal insufficiency and wounds.

### TECHNICAL FIELD

The present invention relates to medicinal peptides and more specifically to a novel peptidomimetic compound 2-amino-6-[(2-amino-5{guanidino}pentanoyl)amino]hexanoic acid and methods for using same.

### BACKGROUND

Cell adhesion to both natural and synthetic substrates is mediated by cell adhesion proteins which are secreted by cells in tissues and are present in the extracellular matrix in vivo. In the matrix cell adhesion proteins are immobilized by virtue of a high affinity interaction with extracellular matrix (ECM) collagens and proteoglycans. Examples of adhesive proteins include fibronectin (FN), vitronectin (VN), collagens, thrombospondin, von Willebrand factor (vWF), elastin, and laminin (LN). Cell adhesion to synthetic surfaces is also mediated by cell adhesion proteins. In this case, dissolved cell adhesion proteins adsorb to the synthetic surface and support cell adhesion and spreading. Several classes of cell-surface receptors have a high affinity for these cell adhesion proteins, and it is by this affinity that cells adhere and spread on the ECM and synthetic substrates. Some receptors bind to a select group of adhesion proteins; whereas, other more promiscuous receptors bind to many adhesion proteins. Fibronectin was the first cell adhesion protein shown to cause adhesion of cells to extracellular substrates. In vitro, serum fibronectin has historically been known as cold-insoluble globulin and is now referred to as plasma fibronectin. Plasma fibronectin must be adsorbed to the culture surface for normal cell attachment and spreading.

To discover the molecular basis of cell adhesion and related cellular activities, several laboratories performed limited proteolysis to isolate cell-adhesive domains in fibronectin. Assignment of cell-binding activity has been based on assays measuring fibroblast attachment or spreading on fragment-coated substrates. Initially peptic cleavage of a 120 kD fibronectin cell-binding fragment and evaluation of its subfragments revealed a 108 amino acid 11.5 kD fragment that supported cell adhesion. From analyses of four synthetic peptides that together spanned the entire 11.5 kD fragment, one active site was localized to a 3.4 kD polypeptide at the C-terminus of the 11.5 kD fragment. Systematic testing of progressively smaller synthetic peptides, based on the 3.4 kD polypeptide sequence, subsequently identified a shorter peptide that now appears to represent the minimal active sequence. The active site contains the tetrapeptide Arg-Gly-Asp-Ser (RGDS). The RGDS sequence interacts with cell-surface fibronectin receptors, as demonstrated by RGDS competitive inhibition of fibroblast cell spreading on fibronectin-coated substrates. Soluble RGDS also inhibited the direct binding of radiolabeled fibronectin to fibroblastic cells in suspension. These competition studies indicated that the RGD sequence is critical for the cell adhesive function of the parent molecule.

After the RGD cell adhesion recognition site in fibronectin was identified, other cell adhesion proteins were examined for related signals. Other proteins with functional RGD sequences include the platelet adhesion proteins fibrinogen and von Willebrand factor, type I collagen, vitronectin, osteopontin, and laminin. These findings imply that RGD is a ubiquitous cell adhesion signal.

RGD-directed cell-surface receptors for various cell adhesion proteins from many cell types were separated using affinity chromatography on Sepharose carrying the appropriate, covalently bound, adhesion protein. Cell-surface adhesion receptors from cell extracts specifically bound to these columns and were eluted with RGD-containing peptide solutions. Fibronectin binds to a receptor that was a heterodimer with a 160 kD α-subunit and a 140 kD β-subunit. Similar affinity chromatography experiments have yielded distinct two-dimer receptors specific for vitronectin and a platelet receptor with affinities for fibrinogen and fibronectin. The heterodimeric structure was characteristic of RGD-directed receptors, with x-subunits ranging between 140 and 160 kD and β-subunits ranging between 90 and 140 kD. These RGD receptors, known as integrins, form the integrin superfamily of cell-surface adhesion proteins.

The integrin superfamily includes cell-surface receptors for both cell-substrate and cell-cell adhesion. Integrins span the cell membrane and have one α subunit and one P subunit. Eighteen distinct α subunits and eight distinct β subunits are currently known, and 24 αβ combinations have been observed. Integrin complexes containing β1 and β3 subunits generally are involved in cell adhesion to the extracellular matrix, while the β2 integrins are involved in cell-cell adhesion. The set of integrins expressed by different cell types varies greatly. Mammalian cells express from two to ten different integrins, depending on the cell type, and provide a means by which the cell senses its local environment and responds to changes in extracellular matrix composition and topography. Initially, integrins were identified as cell-surface adhesion receptors mechanically linking the cytoskeleton to the extracellular matrix or to other cells. Now integrins are known to regulate cellular adhesion, migration, invasion, proliferation, angiogenesis, bone resorption, apoptosis, and gene expression.

With the discovery of integrins and RGD as the major receptor-ligand system for promoting cell-ECM interactions and some cell-cell interactions, many fundamental cellular events were found to be regulated by integrin-mediated cell attachment to ECM, e.g., cell migration, growth, differentiation, and apoptosis. This resulted in the development of reagents containing or mimicking the RGD sequence: Immobilized on a solid surface, they promote cell adhesion; in soluble form, they inhibit cell adhesion. Therapeutic applications for soluble RGD-containing and RGD-mimetic reagents include tumor growth, cancer metastasis, thrombosis, occlusive cardiovascular disease, osteoporosis, rheumatoid arthritis, diabetic retinopathy, renal failure, inflammation, infection, and wound healing. ROD peptide surface coatings on biomedical implant materials have been shown to promote faster and more complete tissue integration at the implant/tissue interface and reduce the foreign body response.

Recent advances in cell/ECM adhesion research have provided important insights into the role of integrin/matrix interactions in disease processes and have lead to the development of biotherapeutics. Candidate reagents for biotherapeutic applications include monoclonal antibodies that block integrin function and RGD peptides or peptidomimetics. However, antibodies are rapidly degraded in the gut. Pepidomimetics are of particular interest because they have low immunogenicity and are resistant to protease/peptidase-mediated degradation. The major disadvantage to peptidomimetics is that they are difficult to synthesize.

### SUMMARY OF INVENTION

In one embodiment, there is disclosed a composition including 2-amino-6-[(2-amino-5{guanidine}pentanoyl)amino]hexanoic acid (AAGPAHA).

In a different embodiment, there is a method of synthesizing an RGD peptidomimetic. The method has the steps of combining equal equivalents of Fmoc-Arg(Pbf)-OH and N-[(dimethylamino)-1H-1,2,3-triazolo[4,5-b]pyridine-1-ylmethylene]-N-methylmethanaminium hexafluorophosphate N-oxide (HATU) and dissolving in 0.5 M diisopropylethylamine in dimethylformamide (DMF); preparing Boc-Lys (Fmoc)-resin by soaking in 20% piperidine and DMF; combining the mixture of Arg and HATU with the Boc-Lys-resin and allowing it to react; and purifying the reaction mixture by washing with DMF and 20% piperidine.

In another embodiment, a method of encouraging cell attachment in cell culture has the steps of providing a cell culture substrate and of adding AAGPAHA to the cell culture substrate.

In yet another embodiment, there is provided an implant that resists cellular interaction in the body. The implant is coated with AAGPAHA.

In another embodiment, a biomaterial suitable for implantation is made capable of resisting cellular interaction after implantation by coating with AAGPAHA.

In another embodiment, there is disclosed a pharmaceutical composition including AAGPAHA and a pharmaceutical excipient.

In yet another embodiment, a method of treating cancer in an individual suffering therefrom has the steps of providing the pharmaceutical composition with AAGPAHA and administering the pharmaceutical composition to the individual.

In yet another embodiment, a method of treating thrombosis in an individual suffering therefrom has the steps of providing the pharmaceutical composition with AAGPAHA and administering the pharmaceutical composition to the individual.

In yet another embodiment, a method of treating osteoporosis in an individual suffering therefrom has the steps of providing the pharmaceutical composition with AAGPAHA and administering the pharmaceutical composition to the individual.

In yet another embodiment, a method of treating retinopathy in an individual suffering therefrom has the steps of providing the pharmaceutical composition with AAGPAHA and administering the pharmaceutical composition to the individual.

In yet another embodiment, a method of treating renal insufficiency in an individual suffering therefrom has the steps of providing the pharmaceutical composition with AAGPAHA and administering the pharmaceutical composition to the individual.

In yet another embodiment, a method of treating wounds in an individual having at least one wound has the steps of providing the pharmaceutical composition with AAGPAHA and administering the pharmaceutical composition to the individual.

### BRIEF DESCRIPTION OF THE DRAWINGS

FIG. 1 is a schematic showing solid phase synthesis of 2-amino-6-[(2-amino-5{guanidino}pentanoyl)amino]hexanoic acid (AAGPAHA).

FIG. 2 is a schematic showing amination of tissue culture plastic.

FIG. 3 is a schematic showing the reaction scheme for the surface immobilization of dextran to amine-bearing tissue culture plastic.

FIG. 4 is a schematic showing the reaction scheme for the surface immobilization of peptides to dextran-coated tissue culture plastic surfaces.

FIG. 5 is a bar graph showing the portion of 3T3 cell coverage of tissue culture plastic coated with (from left to right) dextran, AAGPAHA and the RGD peptide.

FIGS. 6A, 6B and 6C are photomicrographs (at 100× magnification) showing adherent cells on dextran (none), AAGPAHA and the RGD peptide, respectively.

FIG. 7 is a bar graph showing the portion of 3T3 cell coverage of tissue culture plastic coated with AAGPAHA (two left bars) and RGD peptide (two right bars). The second bar from the left shows decreased cell coverage of AAGPAHA surface in the presence of soluble RGD peptide that competes with AAGPAHA. The far right bar shows the cell coverage of RGD peptide surface in the presence of soluble AAGPAHA, which reduced cell coverage to zero.

FIGS. 8A, 8B, 8C and 8D are photomicrographs (100× magnification) of adherent cells for each substrate with or without soluble RGD peptide or AAGPAHA. FIG. 8A shows 3T3 cell adhesion and spreading on the AAGPAHA substrate. FIG. 8B shows reduced 3T3 cell adhesion on the same substrate in the presence of the RGD peptide. FIG. 8C shows 3T3 cell adhesion and spreading on the RGD peptide substrate. This response is effectively inhibited when soluble AAGPAHA is added, as shown in FIG. 8D.

FIGS. 9A and 9B show mass spectrometry results for the RGD peptide before (FIG. 9A) and after (FIG. 9B) trypsin exposure (1 h, 37° C.). Arrows indicate the 588 nm peak for the RGD peptide.

FIGS. 10A and 10B show mass spectrometry results for AAGPAHA before (FIG. 10A) and after (FIG. 10B) trypsin exposure (1 h, 37° C.). Arrows indicate the 303 nm mass peak of AAGPAHA.

FIGS. 11A and 11B show mass spectrometry results for the RGD peptide before (FIG. 11A) and after (FIG. 11B) prolonged trypsin exposure (16 h, 25° C.). Arrows indicate the 588 nm peak for the RGD peptide.

FIGS. 12A and 12B show mass spectrometry results for AAGPAHA before (FIG. 12A) and after (FIG. 12B) prolonged trypsin exposure (16 h, 25° C.). Arrows indicate the 303 nm mass peak of AAGPAHA.

### DETAILED DESCRIPTION

The present invention is a novel RGD peptidomimetic that has a simple and efficient synthesis protocol. The present invention relates to a novel peptidomimetic compound 2-amino-6-[(2-amino-S{guanidino}pentanoyl)amino]hexanoic acid (AAGPAHA) having the same integrin-binding activities as Arg-Gly-Asp (RGD)-containing synthetic peptides, but is very stable to proteolytic degradation in comparison to proteolytically unstable peptides. Thus, this novel compound would be immune to gastric acidity and destruction. This novel non-peptide mimetic has broad therapeutic utility of the RGD peptides combined with higher stability and therapeutic efficacy. The RGD peptide mimetic also is simple to synthesize in comparison to the state-of-the-art in the design and synthesis of other RGD peptide mimetics. The structural formula of this compound is as follows, with the native RGD peptide sequence (RGD) shown below as a reference:

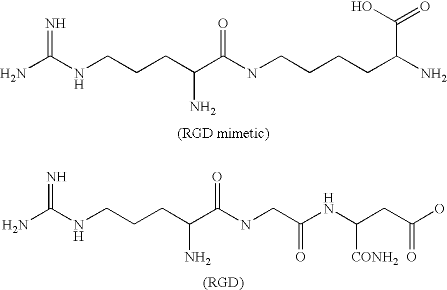

The above composition, which exemplifies the invention, can be varied in ways that are well known in the art. For example, a variety of oxidation reactions are known in the art and are applicable for use in conjunction with the invention. Suitable oxidizing reactants include nitroxyl radicals, nitrogen dioxide and tetroxide, and hydrogen peroxide. Alternatively, the oxidation may also be effectuated enzymatically or via electrolytic methods. Suitable such reactions are disclosed in art.

Salts of the compounds of the present invention may comprise acid addition salts derived from a nitrogen on a substituent in the inventive compound. Salts encompassed within the term “pharmaceutically acceptable salts” refer to non-toxic salts of the compounds of this invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention. The compounds of this invention may be made by a variety of methods, including standard chemistry.

Pharmaceutical Compositions

In yet another aspect of the present invention, provided are pharmaceutical compositions of AAGPAHA. Such pharmaceutical compositions may be administered by injection, or by oral, pulmonary, nasal or other forms of administration. In general, comprehended by the invention are pharmaceutical compositions comprising effective amounts of a cyclic or derivative product, of the invention together with pharmaceutically acceptable diluents, preservatives, solubiilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., thimerosal, benzyl alcohol) and bulking substances (e.g., lactose, mannitol); incorporation of the material into particulate preparations of polymeric compounds such as polylactic acid, polyglycolic acid, etc., or into liposomes. Hylauronic acid may also be used. Such compositions may influence the physical state, stability, rate of in vivo release, and rate of in vivo clearance of the present compound and derivatives. See, e.g., REMINGTON'S PHARMACEUTICAL SCIENCES, 18^{th} Ed. (1990, Mack Publishing Co., Easton, Pa., pp 1435-1712) which are herein incorporated by reference. The compositions may be prepared in liquid form, or may be in dried powder, such as lyophilized form.

Oral delivery is contemplated for use herein. AAGPAHA has been specially invented for the oral route of administration. Oral solid dosage forms are described generally in REMINGTON'S PHARMACEUTICAL SCIENCES, 18^{th} Ed. (1990 Mack Publishing Co., Easton, Pa., at Chapter 89), which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). In general, the formulation includes AAGPAHA (or chemically modified forms thereof) and inert ingredients which allow for protection against the stomach environment, and for release of the biologically active material in the intestine.

Also specifically contemplated are oral dosage forms of the above derivatized compound. The compound may be further chemically modified so that oral delivery of the derivative is efficacious. Generally, the chemical modification contemplated is the attachment of at least one moiety to the component molecule itself, where said moiety permits (a) inhibition of proteolysis; and (b) uptake into the bloodstream from the stomach or intestine. Also desired is the increase in overall stability of the component or components and increase in circulation time in the body. An example of such a moiety is polyethylene glycol (PEG).

For the compound (or derivative), the location of release may be the stomach, the small intestine (the duodenum, the jejunum, or the ileum), or the large intestine. One skilled in the art has available formulations that will not dissolve in the stomach, yet will release the material in the duodenum or elsewhere in the intestine. Preferably, the release will avoid the deleterious effects of the stomach environment, either by protection of the protein (or derivative) or by release of the biologically active material beyond the stomach environment, such as in the intestine.

The compound can be included in the formulation as fine multi-particulates in the form of granules or pellets of particle size about 1 mm. The formulation of the compound for capsule administration could also be as a powder, lightly compressed plugs or even as tablets. The compound could be prepared by compression.

One may dilute or increase the volume of the compound with an inert material. These diluents could include carbohydrates, especially mannitol, a-lactose, anhydrous lactose, cellulose, sucrose, modified dextrans and starch. Certain inorganic salts may be also be used as fillers, including calcium triphosphate, magnesium carbonate and sodium chloride. Some commercially available diluents are Fast-Flo, Emdex (E. Mendell Co., Inc., Patterson, N.Y.), STA-Rx 1500 (A.E. Staley Mfg. Co. Corp., Decatur, Ill.), Emcompress (E. Mendell Co.) and Avicel microcrystalline cellulose (FMC Corp., Philadelphia, Pa.).

Disintegrants may be included in the formulation of the compound as a solid dosage form. Materials used as disintegrants include, but are not limited to starch, including the commercial disintegrant based on starch, Explotab (E. Mendell Co.). Binders also may be used to hold the therapeutic agent together to form a hard tablet and include materials from natural products such as acacia, tragacanth, starch and gelatin.

An anti-frictional agent may be included in the formulation of the compound to prevent sticking during the formulation process. Lubricants may be used as a layer between the therapeutic and the die wall. Glidants that might improve the flow properties of the drug during formulation and to aid rearrangement during compression also might be added. The glidants may include starch, talc, pyrogenic silica and hydrated silicoaluminate.

In addition, to aid dissolution of the therapeutic into the aqueous environment a surfactant might be added as a wetting agent. Additives which potentially enhance uptake of the compound (or derivative) are, for instance, the fatty acids oleic acid, linoleic acid and linolenic acid.

Nasal delivery of the AAGPAHA or derivative thereof is also contemplated. Nasal delivery allows the passage of the protein to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution to a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation forms a spray when squeezed. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

Transdermal Administration

Various and numerous methods are known in the art for transdermal administration of a drug, e.g., via a transdermal patch. Transdermal patches are described in, for example, U.S. Pat. No. 5,407,713, issued Apr. 18, 1995, to Rolando et al.; U.S. Pat. No. 5,352,456, issued Oct. 4, 1994, to Fallon et al.; U.S. Pat. No. 5,332,213 issued Aug. 9, 1994, to D'Angelo et al.; U.S. Pat. No. 5,336,168, issued Aug. 9, 1994, to Sibalis; U.S. Pat. No. 5,290,561, issued Mar. 1, 1994, to Farhadieh et al.; U.S. Pat. No. 5,254,346, issued Oct. 19, 1993, to Tucker et al.; U.S. Pat. No. 5,164,189, issued Nov. 17, 1992, to Berger et al.; U.S. Pat. No. 5,163,899, issued Nov. 17, 1992, to Sibalis; U.S. Pat. Nos. 5,088,977 and 5,087,240, both issued Feb. 18, 1992, to Sibalis; U.S. Pat. No. 5,008,110, issued Apr. 16, 1991, to Benecke et al.; and U.S. Pat. No. 4,921,475, issued May 1, 1990, to Sibalis, the disclosure of each of which is incorporated herein by reference in its entirety.

It can be readily appreciated that a transdermal route of administration may be enhanced by use of a dermal penetration enhancer, e.g., such as enhancers described in U.S. Pat. No. 5,164,189 (supra), U.S. Pat. No. 5,008,110 (supra), and U.S. Pat. No. 4,879,119, issued Nov. 7, 1989, to Aruga et al., the disclosure of each of which is incorporated herein by reference in its entirety.

Pulmonary Delivery

Also contemplated herein is pulmonary delivery of the pharmaceutical compositions of the present invention. A pharmaceutical composition of the present invention is delivered to the lungs of a mammal while inhaling and traverses across the lung epithelial lining to the blood stream. Other reports of this include Adjei et al. [Pharmaceutical Research, 7:565-569 (1990); Adjei et al., Intl J Pharmaceutics, 63:135-144 (1990) (leuprolide acetate); Braquet et al., J Cardiovascular Pharm, 13(suppl. 5):143-146 (1989(endothelin-1); Hubbard et al., Ann Internal Medicine, Vol. III, pp. 206-212 (1989) (α1-antitrypsin); Smith et al., J Clin Invest, 84:1145-1146 (1989) (α-1-proteinase); Oswein et al., “Aerosolization of Proteins”, Proceedings of Symposium on Respiratory Drug Delivery II, Keystone, Colo., March, (1990) (recombinant human growth hormone); Debs et al., J Immunol, 140:3482-3488 (1988) (interferon γ and tumor necrosis factor α); Platz et al., U.S. Pat. No. 5,284,656 (granulocyte colony stimulating factor)]. A method and composition for pulmonary delivery of drugs for systemic effect is described in U.S. Pat. No. 5,451,569, issued Sep. 19, 1995, to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. With regard to construction of the delivery device, any form of aerosolization known in the art, including but not limited to spray bottles, nebulization, atomization or pump aerosolization of a liquid formulation, and aerosolization of a dry powder formulation, can be used in the practice of the invention.

All such devices require the use of formulations suitable for the dispensing of pharmaceutical composition of the present invention (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. A chemically modified pharmaceutical composition of the present invention may also be prepared in different formulations depending on the type of chemical modification or the type of device employed.

Formulations suitable for use with a nebulizer, either jet or ultrasonic, may typically comprise pharmaceutical composition of the present invention (or derivative) dissolved in water at a concentration of, e.g., about 0.01 to 25 mg of biologically active ingredients of a pharmaceutical composition of the present invention per mL of solution. The formulation may also include a buffer and a simple sugar (e.g., for stabilization and regulation of osmotic pressure of a pharmaceutical composition of the present invention). The nebulizer formulation may also contain a surfactant, to reduce or prevent surface induced aggregation of the pharmaceutical composition of the present invention caused by atomization of the solution in forming the aerosol.

The liquid aerosol formulations contain a pharmaceutical composition of the present invention and a dispersing agent in a physiologically acceptable diluent. The dry powder aerosol formulations of the present invention consist of a finely divided solid form of a pharmaceutical composition of the present invention and a dispersing agent. With either the liquid or dry powder aerosol formulation, the formulation must be aerosolized. That is, it must be broken down into liquid or solid particles in order to ensure that the aerosolized dose actually reaches the mucous membranes of the nasal passages or the lung. The term “aerosol particle” is used herein to describe the liquid or solid particle suitable for nasal or pulmonary administration, i.e., that will reach the mucous membranes. Other considerations, such as construction of the delivery device, additional components in the formulation, and particle characteristics are important. These aspects of nasal or pulmonary administration of a drug are well known in the art, and manipulation of formulations, aerosolization means and construction of a delivery device require at most routine experimentation by one of ordinary skill in the art.

Often, the aerosolization of a liquid or a dry powder formulation for inhalation into the lung will require a propellant. The propellant may be any propellant generally used in the art. Specific non-limiting examples of such useful propellants are a hydrocarbon, including trifluoromethane, dichlorodifluoromethane, dichlorotetrafluoroethanol, and 1,1,1,2-tetrafluoroethane, or combinations thereof.

Systems of aerosol delivery, such as the pressurized metered dose inhaler and the dry powder inhaler are disclosed in Newman, S. P., Aerosols and the Lung, Clarke, S. W. and Davia, D. Eds., pp 197-22 and can be used in connection with the present invention.

Liquid Aerosol Formulations

The present invention provides aerosol formulations and dosage forms. In general, such dosage forms contain a pharmaceutical composition of the present invention in a pharmaceutically acceptable diluent. Pharmaceutically acceptable diluents include but are not limited to sterile water, saline, buffered saline, dextrose solution, and the like.

The formulation may include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half life for clearance. Such macromolecules include but are not limited to Soya lecithin, oleic acid and sorbitan trioleate, with sorbitan trioleate preferred.

The formulations of the present embodiment may also include other agents useful for pH maintenance, solution stabilization, or for the regulation of osmotic pressure.

Aerosol Dry Powder Formulations

It is also contemplated that the present aerosol formulation can be prepared as a dry powder formulation comprising a finely divided powder form of pharmaceutical composition of the present invention and a dispersant. Formulations for dispensing from a powder inhaler device will comprise a finely divided dry powder containing pharmaceutical composition of the present invention (or derivative) and may also include a bulking agent, such as lactose, sorbitol, sucrose, or mannitol in amounts which facilitate dispersal of the powder from the device, e.g., 50 to 90% by weight of the formulation. The pharmaceutical composition of the present invention (or derivative) should most advantageously be prepared in particulate form with an average particle size of less than 10 mm (or microns), most preferably 0.5 to 5 mm, for most effective delivery to the distal lung.

Methods of Treatment, Methods of Preparing a Medicament

In yet another aspect of the present invention, methods of treatment and manufacture of a medicament are provided. Conditions alleviated or modulated by the administration of the present derivatives are those indicated above.

Dosages

For all of the above molecule, as further studies are conducted, information will emerge regarding appropriate dosage levels for treatment of various conditions in various patients; and the ordinary skilled worker, considering the therapeutic context, age and general health of the recipient, will be able to ascertain proper dosing. In addition, where appropriate, the size of the tumor may be relevant. In general, the dosage is between about 0.01 mg/day and 2 grams per day. The lower limit of the range is selected from 0.1, 1.0, 10.0, 60, 75, 100 or 500 mg/day. The upper limit of the range is selected from 1.5, 1.2, 1.0, 0.9, 0.8, 0.7, 0.6, 0.5, 0.4, 0.3 or 0.2 grams per day.

A subject in whom administration of AAGPAHA or an active variant thereof is an effective therapeutic regimen is preferably a human, but can be any animal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as, but not limited to, bovine, equine, caprine, ovine, and porcine subjects, wild animals (whether in the wild or in a zoo), research animals, such as mice, rats, rabbits, goats, sheep, pigs, dogs, cats, etc., avian species, such as chickens, turkeys, songbirds, etc., i.e., for veterinary medical use.

In vitro studies were undertaken to assess both the cell adhesive properties of surface immobilized 2-amino-6-[(2-amino-5 {guanidino}pentanoyl)amino]hexanoic acid (AAGPAHA) peptidomimetic and the counteradhesive integrin antagonist activity of soluble AAGPAHA. AAGPAHA was covalently immobilized on dextran-coated tissue culture plastic utilizing methods earlier developed for cell adhesion peptides. Cell adhesion was on surface-immobilized AAGPAHA and compared to surface-immobilized peptide GRGDSP. Other experiments were designed to assess the effect of soluble AAGPAHA on cell adhesion to GRGDSP to determine whether soluble AAGPAHA competitively inhibited integrin-RGD mediated cell adhesion. A similar experimental series was conducted to determine whether soluble, integrin-binding GRGDSP competitively inhibited cell adhesion to surface immobilized AAGPAHA. Experimental methods and results are discussed below

### EXAMPLES

### Example 1

### Synthesis of 2-amino-6-[(2-amino-5{guanidino}pentanoyl)amino]hexanoic acid (AAGPAHA)

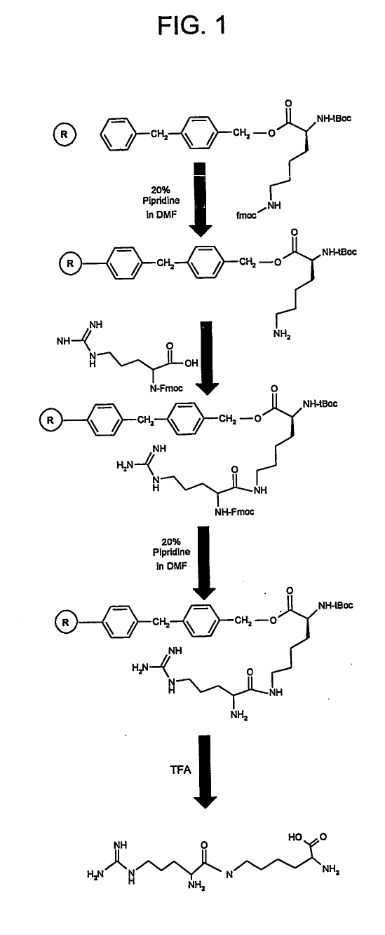

Solid phase synthesis was used to prepare AAGPAHA. FIG. 1 schematically depicts the synthetic scheme where initially Wang resin-bound (R) lysine was covalently linked to arginine by forming an amide bond with the ε amino group on lysine with the α carboxy group from arginine.

Five grams (0.5 mmol/g) of Boc-Lys (Fmoc)-Wang resin (Advanced ChemTech, Louisville, Ky., Cat # SK5151) was soaked in 20% piperidine in N,N-dimethylformamide (DMF, Advanced ChemTech, Cat # RC8206) for 30 min to swell and also to remove the Fmoc group. The resin was then washed with DMF several times to remove all piperidine (at least three times). Two equivalents (3.244 g) of Fmoc-Arg (Pbf)-OH (Advanced ChemTech, Cat # FR2136) together with 2 equivalents of N-[(dimethylamino)-1H-1,2,3-triazolo[4,5-b]pyridine-1-ylmethylene]-N-methylmethanaminium hexafluorophosphate N-oxide (HATU, 1.901 g) were combined and dissolved in 0.5 M diisopropylethylamine (DIPEA) in DMF. (1 equivalent=2.5 mmole, 2 equivalent=5 mmole). The total volume of the solvent was 21.8 ml. This solution was added to the washed resin and reacted for 45 min to 1 hr. The resin was then washed with DMF to remove residual unreacted Arg and HATU. Next 20% piperidine in DMF was added to remove Fmoc from resin-bound Arg α-amino groups. The resin was then allowed to stand for 30 min and followed by these washes: a) DMF to remove piperidine, and b) CH₂Cl₂ (dichloromethane) to remove DMF. The resins were then dried overnight under vacuum. To cleave the product from resin, trifluoroacetic acid (TFA, Advanced ChemTech, Cat # RA8402) containing triisopropylsilane and ethanedithiol (in the ratio of 95:2.5:2.5) was added and left for 4 hrs. The cleaved product AAGPAHA was precipitated with ether and dried under vacuum overnight.

### Example 2

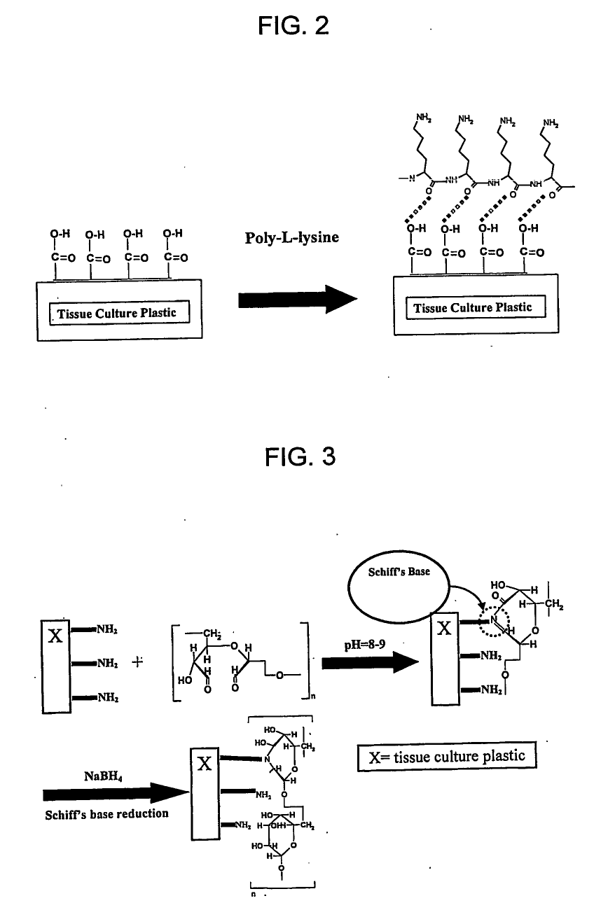

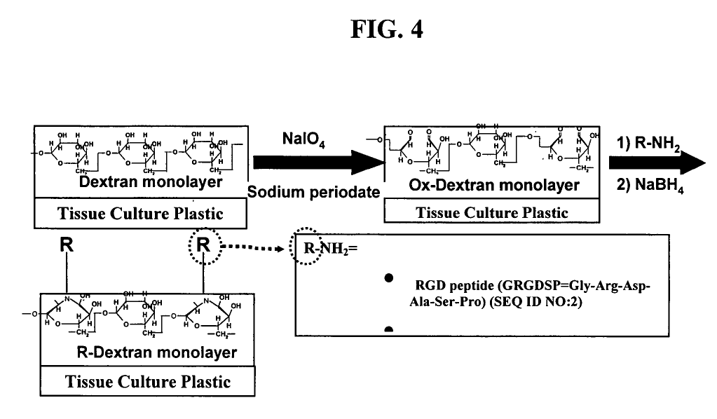

For this study, we immobilized AAGPAHA and the peptide GRGDSP on dextran-coated tissue culture plastic dishes in a series of steps shown in FIGS. 2-4. Multi-well cell culture dishes were first surface-aminated by adsorbing poly-lysine (FIG. 2). Dextran was then immobilized on these surfaces (FIG. 3) using reported methods (Massia, Biomaterials 21:2753-61, 2000). AAGPAHA and GRGDSP were then surface-immobilized on dextran-coated tissue culture plastic (FIG. 4) using reported methods (Massia, J Biomed Mater Res 56:390-99, 2001). Dextran substrates were activated by oxidation of the glucose subunits (Glc) with sodium metaperiodate to convert Glc subunits to cyclic hemiacetal structures. Hemiacetal-containing subunits were then reacted with N-terminal amines of peptides forming an amine linkage between peptides and surface-immobilized dextran.

Periodate Oxidation of Dextran

Dextran was oxidized to produce aldehyde groups via standard periodate methods (FIG. 3). One gram of dextran (M.W. 40 kDa, Sigma, St. Louis, Mo.) was first dissolved in 30 ml of deionized water. Sodium meta periodate (NaIO₄, 0.1 M) was prepared for immediate use. This NaIO₄ solution was then added to the solution of dextran to make a 50% molar ratio of NaIO₄ to dextran expressed as moles of glucose monomer. The reaction mixture was stirred at 4° C. overnight and protected from light by covering the reaction flask with aluminum foil. The solution was then purified by precipitation of unreacted periodate and iodate products using an equimolar aqueous solution of BaCl₂. The purified oxidized dextran solution was then lyophilized and stored (if not immediately used) at 4° C. in a 50 mL conical centrifuge tube protected from light. The product was analyzed by Fourier transform infrared spectroscopy (FT-IR). The results showed a peak at 1700 nm, indicating the aldehyde groups within the dextran chain (not shown).

Covalent Coupling of Oxidized Dextran to Surface-Aminated Cell Culture Dishes.

Multi-well cell culture dishes were first surface-aminated by immediate immersion in 0.01% aqueous poly-L-lysine (PLL) solution then incubated overnight (FIG. 2). Oxidized dextran, prepared as described above, was dissolved in 0.2 M sodium phosphate buffer (pH 9, 0.02 g/mL). Immediately following surface amination procedures, oxidized dextran solution (2 mL) was added to six-well multiwell dishes containing surface-aminated substrates (FIG. 3) where the glucose units formed Schiff bases with the lysine amines. The substrates were allowed to incubate at room temperature for 16 hrs on a rocker platform and protected from light. Following incubation, the reaction mixture was decanted from the culture wells and replaced by fresh 0.1 M solution of sodium borohydride, NaBH₄ to reduce Schiff bases and to quench any free unreacted aldehyde groups present on the oxidized dextran chain. The substrates were allowed to incubate for 2 hrs on the rocker platform. The NaBH₄ solution was then decanted and the substrates were rinsed gently several times with deionized water to remove unbound dextran (FIG. 3).

Covalent Coupling of GRGDSP and AAGPAHA.

Surface grafting of peptide GRGDSP and peptidomimetic AAGPAHA to dextran-coated substrates was achieved via previously reported methods (Massia, 2001). Dextran-coated substrates committed for peptide or peptidomimetic grafting were oxidized with 0.1 M sodium periodate, for 1 hr at room temperature to activate substrate surfaces for covalent immobilization of peptides (FIG. 4). Following surface oxidation, samples were rinsed with deionized water and then peptide or peptidomimetic stock solutions (0.1 mg/mL in 0.2 M dibasic sodium phosphate, pH 9.0) were added to each sample well. For 24 hrs a rocker table agitated the plates that were protected from light with a tin foil covering. Culture well substrates were decanted and rinsed with deionized water at the end of the 24 hr duration. Following peptide/peptidomimetic coupling, the substrates were incubated in dibasic sodium phosphate (0.2 M, pH 9.0) containing 0.1 M sodium borohydride, NaBH₄, to reduce Schiff bases formed and to quench any free unreacted aldehyde groups present (FIG. 4). The substrates were allowed to incubate for 2-3 hrs on the rocker platform. The substrates were rinsed with PBS and immediately were employed for in vitro experiments.

### Example 3

### Cell Adhesion Assays

Cell Culture.

The cell line utilized in these studies were 3T3 fibroblasts (ATCC # CRL-6476, Manassas, Va.). 3T3 cells were maintained in Dulbecco's modified Eagle medium (DMEM) with 10% fetal bovine serum (FBS) at 37° C. in saturated humidity. All cell culture media and reagents were obtained from Life Technologies, Inc. (Rockville, Md.).

Cell Adhesion Assay.

Cell area coverage assays were performed to assess the extent of 3T3 cell adhesion and spreading on all substrates. 3T3 cells were seeded on 24-well culture dishes (15,000 cells/well) and incubated for 24 hrs. Following incubation, samples were fixed (3.8% formaldehyde in phosphate buffered saline (PBS) for 5 min. and stained with 0.1% aqueous toluidine blue (5 min). Stained cells were then examined using phase contrast or stereomicroscopy (Leica) at 100× magnification. Three random 100× fields were selected for each substrate for analysis. The extent of cell adhesion was determined for each captured digital image by calculating a percentage of cell area coverage using digital image analysis software. Final data were presented as a percentage of control adhesion.

The percentage of control adhesion was calculated by multiplying the ratio of % area coverage on dextran-coated, peptide-grafted, or peptidomimetic-grafted substrates to % cell area coverage on untreated tissue culture plastic by 100. The average percentage of control adhesion was determined from duplicate independent experiments. Comparisons between sample groups were made using analysis of variance between groups (ANOVA).

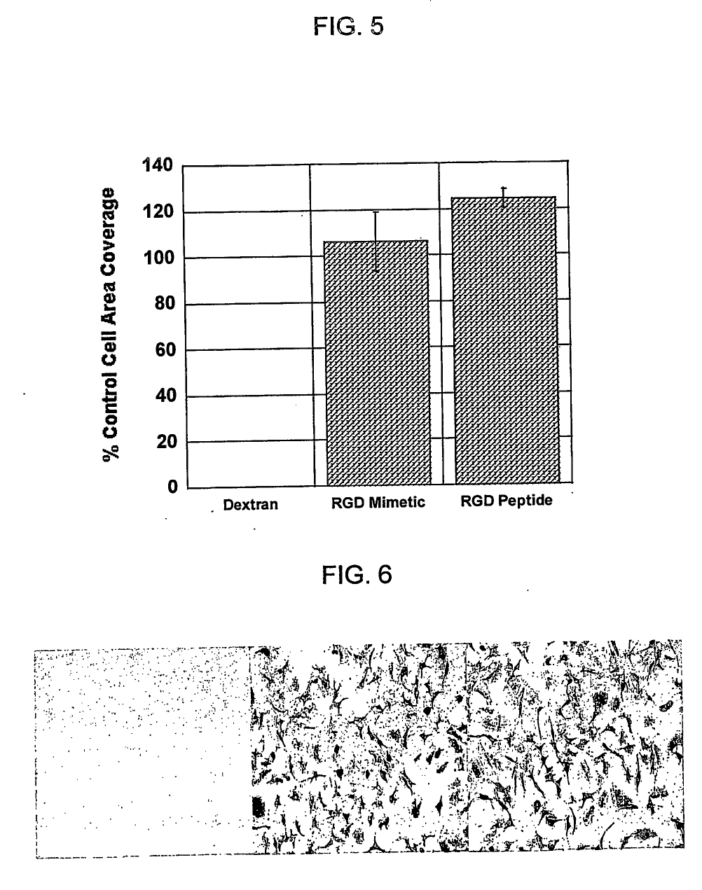

Surface immobilization of dextran on tissue culture wells significantly reduced adhesion and spreading of all cell types (FIG. 5; 0.017±0.03% control). Virtually no cells were observed (FIG. 6A). Surface immobilization of AAGPAHA (the RGD mimetic) promoted extensive cell adhesion and spreading (FIG. 5; 105.9±12.8% control), comparable to substrates containing surface-grafted GRGDSP peptide (FIG. 5; 124.3±4.1% control). FIGS. 6B and 6C show thorough cell coverage of both protein surfaces. These results show that cells adhere to AAGPAHA as effectively as to the RGD peptide.

Cell Adhesion Inhibition Assay.

This assay was performed using methods described above in Cell Adhesion Assay. Cells were seeded on 24-well culture dishes and incubated for 24 hrs. One group of cells seeded on RGD peptide-grafted substrates contained 0.1 mg/mL soluble peptidomimetic in the culture medium. Another group was seeded on peptidomimetic-grafted substrates in the presence of 0.1 mg/mL soluble RGD peptide in the culture medium. The other two groups were seeded on RGD peptide or peptidomimetic substrates without soluble peptide or soluble peptidomimetic. The percentage of control adhesion was determined as described above. The average percentage of control adhesion was determined from duplicate independent experiments. Comparisons between sample groups were made using ANOVA.

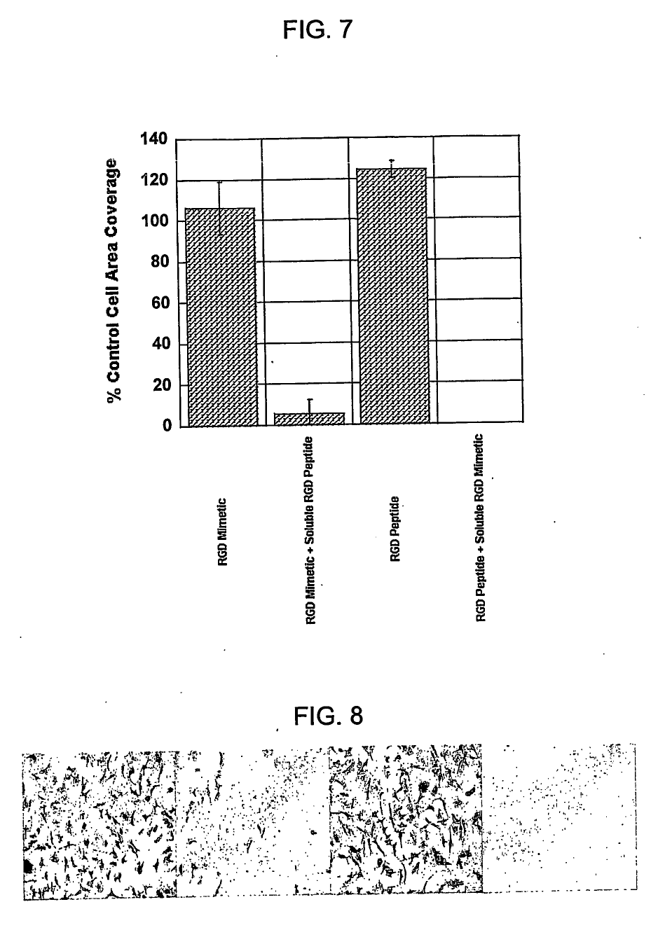

Soluble AAGPAHA peptidomimetic (0.1 mg/mL) completely inhibited 3T3 cell adhesion and spreading on surface-immobilized GRGDSP (FIG. 7; 0.08%±0.6% control). This result indicates that soluble AAGPAHA bound to cell integrins competitively to inhibit integrin-mediated adhesion and spreading, cf., FIG. 8C without soluble AAGPAHA and FIG. 8D with soluble AAGPAHA.

Similarly, soluble GRGDSP peptide (1 mg/mL) completely inhibited cell adhesion and spreading on surface-immobilized AAGPAHA (two left bars of the FIG. 7 graph; 5.7%±6.9% control), cf., FIG. 8A without soluble RGD and FIG. 8B with soluble RGD. This result indicates that cell adhesion to surface-immobilized AAGPAHA peptidomimetic substrates is integrin-mediated and can be inhibited by soluble integrin-binding GRGDSP peptide.

Enzymatic Stability of RGD Peptide and RGD Peptidomimetic in Solution.

GRGDSP and AAGPAHA were mixed with trypsin solution in HEPES buffer (10 mM, pH=8) to a final solution of 0.4 mM peptide and 0.05 mM (0.12%) trypsin and incubated at 37° C. for 1 hr. In a control experiment, both peptides were dissolved in the same buffer without trypsin at the same final concentrations and incubated at 37° C. for 1 hr. Similarly, in another set of experiments, the same samples were incubated at room temperature overnight. At the end of the incubation period, trypsin activity was quenched by adding inhibitor; and the samples were analyzed by MALDI-TOF mass spectrometry to assess the extent of degradation.

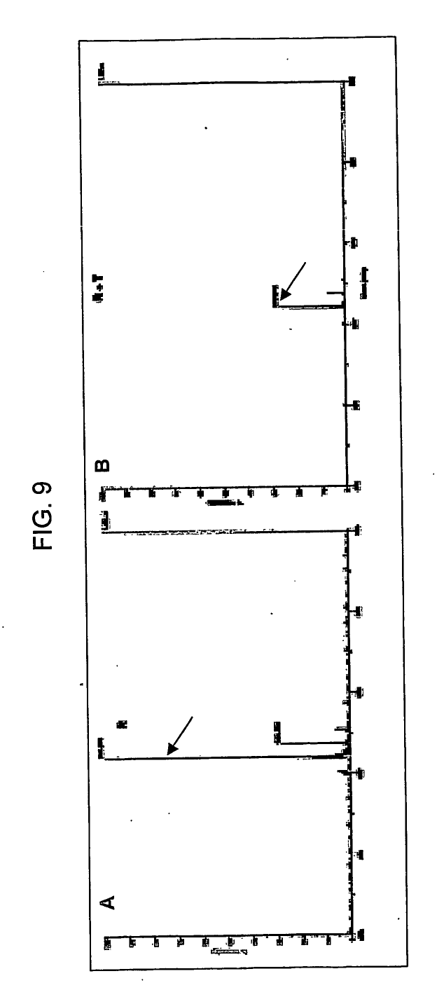

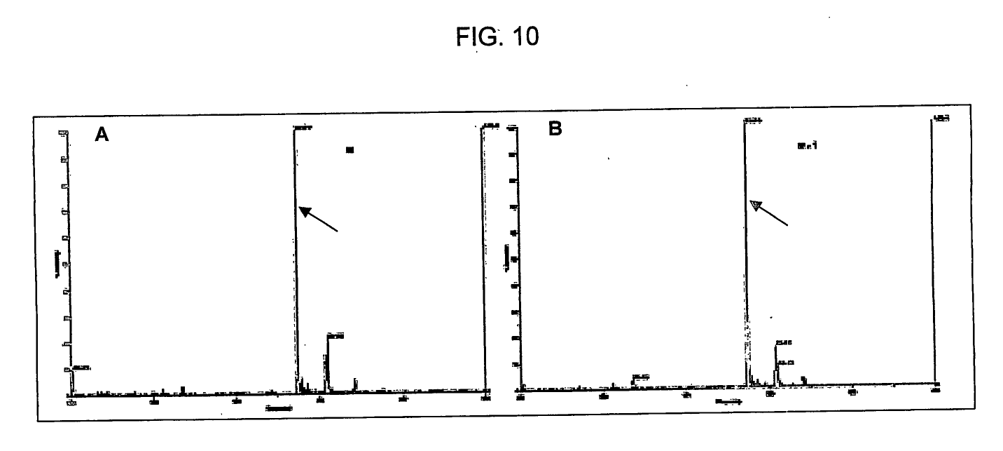

FIG. 9 shows that 588 nm is the mass peak for the RGD peptide before (FIG. 9A) and after (FIG. 9B) trypsinization (1 hr, 37° C.). In FIG. 9A, the RGD peak had an inensity of 1.5×10⁴; after trypsin exposure, FIG. 9B showed an intensity of only 4.0×10³. As can be seen in FIG. 9B at the arrow, there is a sharp decrease (73%) in peak size at mass 588 after treatment with trypsin. In contrast, FIGS. 10A and 10B show no significant change in the mass peak at 303 nm of AAGPAHA after trypsinization (1 hr, 37° C.). In FIG. 10A, the AAGPAHA peak had an intensity of 1.2×10⁴. In FIG. 10B, the AAGPAHA peak intensity was the same.

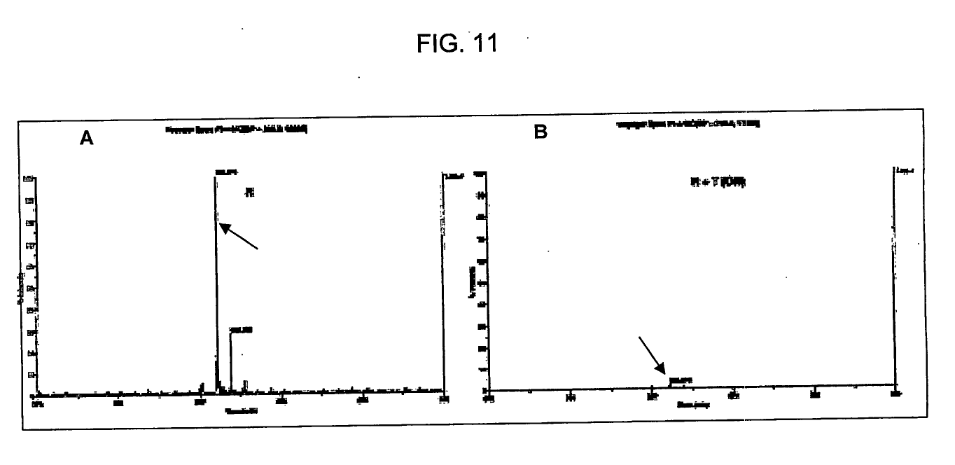

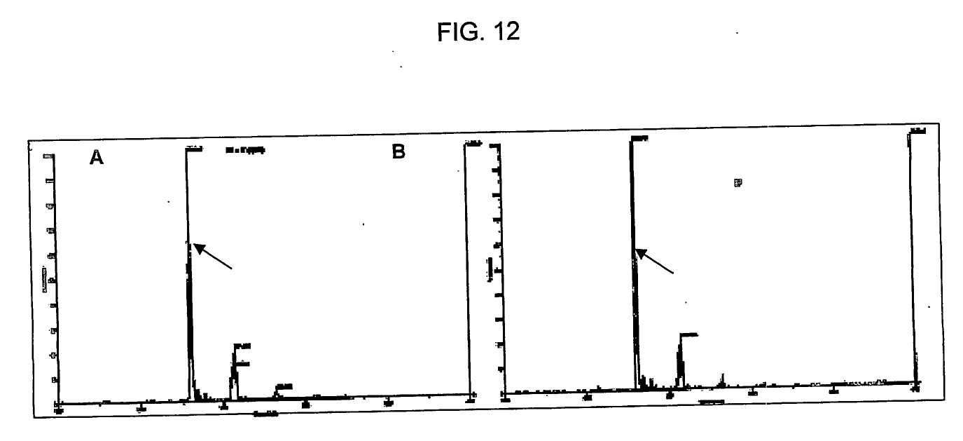

The results obtained from enzymatic treatment at 16 hrs, room temperature for RGD and AAGPAHA are shown in FIGS. 11 and 12, respectively. As can be seen in FIG. 11A, the RGD peak had an intensity of 1.5×10⁴ in comparison to 3.0×10² after trypsinization (FIG. 11B). This is a massive decrease in the 588 nm GRGDSP mass peak, about 98% degradation of peptide. The FIGS. 12A and 12B mass spectra show mass peak intensity for AAGPAHA of 1.2×10⁴, which was the same after 16 hrs, room temperature treatment with trypsin (FIG. 12B), indicating that AAGPAHA is highly resistant to trypsin-mediated degradation.

In conclusion, AAGPAHA is approximately as active as an RGD peptide and is much more resistant to degradation by a major enzyme of the small intestine.

### INDUSTRIAL APPLICABILITY

Commercial applications of this invention include biopharmaceuticals for the treatment of many diseases and abnormal conditions. These include but are not limited to tumor growth, cancer metastasis, thrombosis, occlusive cardiovascular disease, rheumatoid arthritis, osteoporosis, retinopathy (particularly diabetic), renal failure and insufficiency, and wound healing. AAHPAHA can also be used as coatings for biomaterials and implants to block adhesion and activation of the recipient's cells. In the laboratory, this invention also can be used as for cell attachment substrates for cell culture.

The present invention has of necessity been discussed herein by reference to certain specific methods and materials. It is to be understood that the discussion of these specific methods and materials in no way constitutes any limitation on the scope of the present invention, which extends to any and all alternative materials and methods suitable for accomplishing the ends of the present invention.

As any person skilled in the art of designing and testing oligos for gene therapy will recognize from the previous description and from the figures and claims, modifications and changes can be made to the preferred embodiments of the invention without departing from the scope of the invention defined in the following claims.

We claim:

1. The composition comprising 2-amino-6-[(2-amino-5{guanidine}pentanoyl)amino]hexanoic acid (AAGPAHA).

2. A method of synthesizing an RGD peptidomimetic, the method comprising
a. combining equal equivalents of Fmoc-Arg(Pbf)-OH and N-[(dimethylamino)-1H-1,2,3-triazolo[4,5-b]pyridine-1-ylmethylene]-N-methylmethanaminium hexafluoruphosphate N-oxide (HATU) and dissolving in 0.5 M diisopropylethylamine in dimethylformamide (DMF);
b. preparing Boc-Lys (Fmoc)-resin by soaking in 20% piperidine and DMF;
c. combining the mixture of step a with the Boc-Lys-resin and allowing it to react; and
d. purifying the reaction mixture of c by washing with DMF and 20% piperidine.

3. A method of encouraging cell attachment in cell culture, the method comprising
a. providing a cell culture substrate; and
b. adding the compound of claim 1 to the cell culture substrate.

4. An implant resisting cellular interaction after implantation, the implant comprising
a. the implant; and
b. a coating on the implant comprising the composition of claim 1.

5. A biomaterial suitable for implantation, the biomaterial capable of resisting cellular interaction after implantation, the biomaterial comprising
a. the biomaterial; and
b. a coating on the biomaterial comprising the composition of claim 1.

6. A pharmaceutical composition comprising
a. AAGPAHA; and
b. a pharmaceutical excipient.

7. A method of treating cancer in an individual suffering therefrom, the method comprising
a. providing the pharmaceutical composition of claim 6; and
b. administering the pharmaceutical composition to the individual.

8. A method of treating thrombosis in an individual suffering therefrom, the method comprising
a. providing the pharmaceutical composition of claim 6; and
b. administering the pharmaceutical composition to the individual.

9. A method of treating osteoporosis in an individual suffering therefrom, the met-hod comprising
a. providing the pharmaceutical composition of claim 6; and
b. administering the pharmaceutical composition to the individual.

10. A method of treating retinopathy in an individual suffering therefrom, the method comprising
a. providing the pharmaceutical composition of claim 6; and
b. administering the pharmaceutical composition to the individual.

11. A method of treating renal insufficiency in an individual suffering therefrom, the method comprising
a. providing the pharmaceutical composition of claim 6; and
b. administering the pharmaceutical composition to the individual.

12. A method of treating wounds in an individual having at least one wound, the method comprising
a. providing the pharmaceutical composition of claim 6; and
b. administering the pharmaceutical composition to the individual.


SEQUENCE LISTING (2 sequences)

SEQ ID NO: 1 - protein, 4 aa, Artificial
  Synthetic peptide
RGDS

SEQ ID NO: 2 - protein, 6 aa, Artificial
  Synthetic peptide
GRGDSP